(19) United States
(12) Patent Application Publication
(10) Pub. No.: US 2013/0060218 A1
(43) Pub. Date: Mar. 7, 2013

(54) ABSORBENT ARTICLE

(71) Applicants: Jun Kudo, Kagawa (JP); Hideyuki Kinoshita, Kagawa (JP); Yuji Takahashi, Kagawa (JP); Mari Minami, Kagawa (JP)

(72) Inventors: Jun Kudo, Kagawa (JP); Hideyuki Kinoshita, Kagawa (JP); Yuji Takahashi, Kagawa (JP); Mari Minami, Kagawa (JP)

(21) Appl. No.: 13/636,817

(22) PCT Filed: Mar. 23, 2011
(86) PCT No.: PCT/JP2011/057931

§ 371 Date: Nov. 20, 2012

(30) Foreign Application Priority Data
Mar. 23, 2010 (JP) 2010-067086

(51) Int. Cl. A61F 13/511

(52) U.S. Cl. 604/379; 604/378

(57) ABSTRACT

An absorbent article that includes a first absorber. The first absorber is disposed over an excretion opening contact region in the absorbent article and a posterior region. In a width direction of the absorbent article, the first absorber is configured to be longer than a second absorber. The second absorber is not disposed in the excretion opening contact region but disposed in the posterior region. A liquid-penneable sheet is disposed farther inwardly of the skin contact surface side of the absorbent article than the second absorber over the excretion opening contact region and the posterior region. In the width direction, the liquid-penneable sheet is configured to be shorter than the first absorber.

### TECHNICAL FIELD

The present invention relates to an absorbent article.

### BACKGROUND ART

Conventionally, in absorbent articles, there is known a technology in which a main body layer including a topsheet, a backsheet, and a first absorber is arranged, and on a skin contact surface side of the absorbent article of the main body layer, an upper layer including a second absorber is arranged, thereby preventing leakage such as menstrual blood (Japanese Patent Application Publication No. 2006-181218, for example).

For example, in such an absorbent article, the above-described second absorber is disposed in an excretion opening contact region, and is not disposed in an anterior region and a posterior region of the excretion opening contact region.

It is noted that in the absorbent article, the posterior region of the excretion opening contact region is configured to stand upright by way of an elastic member.

### SUMMARY OF INVENTION

### Problem(s) to Be Solved by Invention

The applicant has discovered the following problems in the absorbent article.

In such an absorbent article, in spite of the face that the excretion opening contact region is a location that contacts a largest amount of bodily fluids often, in the excretion opening contact region, the second absorber is disposed immediately below a surface layer of the upper layer on which only a small number of gaps are provided.

Therefore, the absorbent article has a problem that the second absorber cannot instantaneously absorb a large amount of excreted bodily fluids, and the bodily fluids spread over the surface layer of the upper layer, which makes a wearer of the absorbent article feel discomfort and worried about the leakage.

Another problem is that in such an absorbent article, in the posterior region of the excretion opening contact region, the second absorber is not disposed, and thus, on the upper layer, the bodily fluid not absorbed in the excretion opening contact region cannot be absorbed.

Therefore, the present invention has been achieved in view of the above-described problems, and an object thereof is to provide an absorbent article capable of preventing a wearer of the absorbent article from feeling discomfort and being worried about the leakage by efficiently absorbing an excreted bodily fluid by a first absorber of a main body layer and by absorbing a bodily fluid not absorbed by the first absorber by a second absorber of an upper layer.

The first feature of the present invention is summarized in that an absorbent article, including a main body layer including a topsheet, a backsheet, and a first absorber disposed between the topsheet and the backsheet, and a second absorber separated from the main body layer, wherein the first absorber is disposed over an excretion opening contact region in the absorbent article and a posterior region of the excretion opening contact region in a longitudinal direction of the absorbent article, in a width direction of the absorbent article, the first absorber is configured to be longer than the second absorber, the second absorber is disposed farther inwardly of a skin contact surface side of the absorbent article than the main body layer, the second absorber is not disposed in the excretion opening contact region but disposed in the posterior region, a liquid-permeable sheet is disposed farther inwardly of the skin contact surface side of the absorbent article than the second absorber over the excretion opening contact region and the posterior region, and in the width direction, the liquid-permeable sheet is configured to be shorter than the first absorber.

### Advantageous Effect(s) of Invention

As explained above, according to the present invention, it is possible to provide an absorbent article capable of preventing a wearer of the absorbent article from feeling discomfort and being worried about the leakage by efficiently absorbing an excreted bodily fluid by a first absorber of a main body layer and by absorbing a bodily fluid not absorbed by the first absorber by a second absorber of an upper layer.

### BRIEF DESCRIPTION OF DRAWINGS

FIG. 1 is a plan view in which an absorbent article according to an embodiment of the present invention is seen from a skin contact surface side.

FIG. 2 is a view showing a state when the absorbent article according to the embodiment of the present invention is attached to an underwear.

FIG. 3(a) is a cross-sectional view along a line a-a of the absorbent article according to the embodiment shown in FIG. 1, and FIG. 3(b) is a cross-sectional view along a line b-b thereof.

FIG. 4 is an external view of the absorbent article according to the embodiment of the present invention.

FIG. 5 is a diagram for explaining a method of manufacturing the absorbent article according to the embodiment of the present invention.

FIG. 6 is a diagram for explaining the operation and effect of the absorbent article according to the embodiment of the present invention.

### DESCRIPTION OF EMBODIMENTS

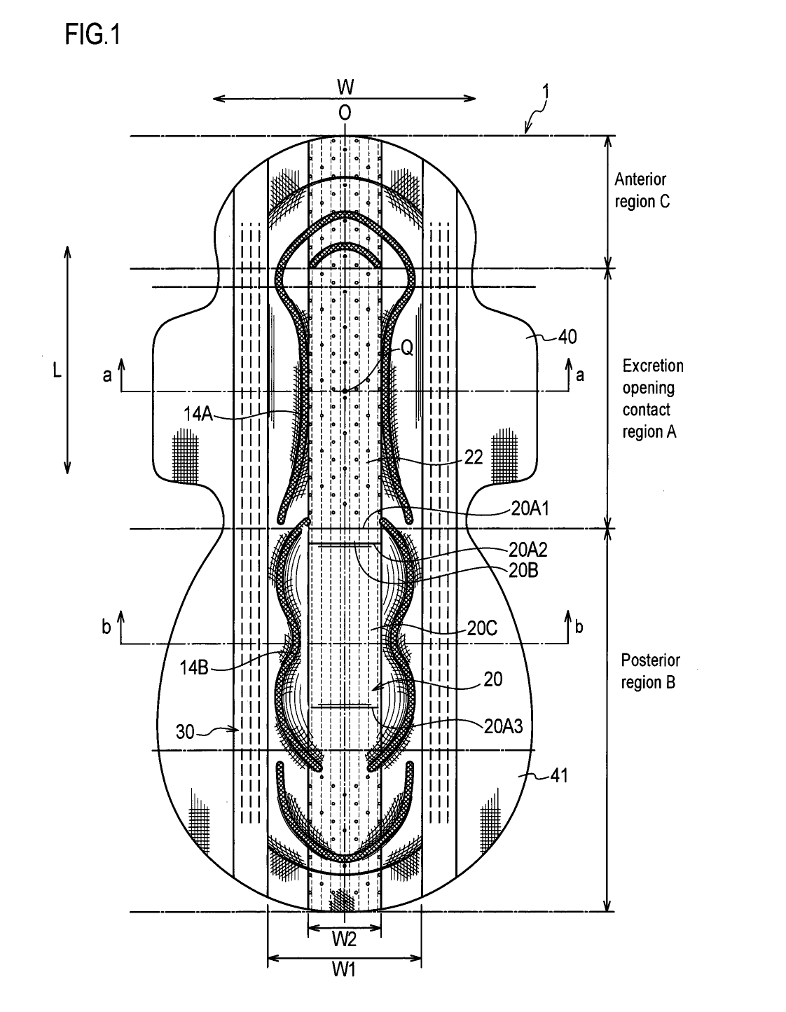

With reference to FIG. 1 to FIG. 6, an absorbent article 1 according to an embodiment of the present invention will be explained. For example, the absorbent article 1 according to this embodiment is a paper diaper, an incontinent pad, and a sanitary napkin, etc.

As shown in FIG. 1, the absorbent article 1 is configured by an excretion opening contact region A, a posterior region B, and an anterior region C.

Relative to the excretion opening contact region A, the posterior region B is disposed at a rearward side of a longitudinal direction L of the absorbent article 1. Relative to the excretion opening contact region A, the anterior region C is disposed at a forward side of the longitudinal direction L of the absorbent article 1.

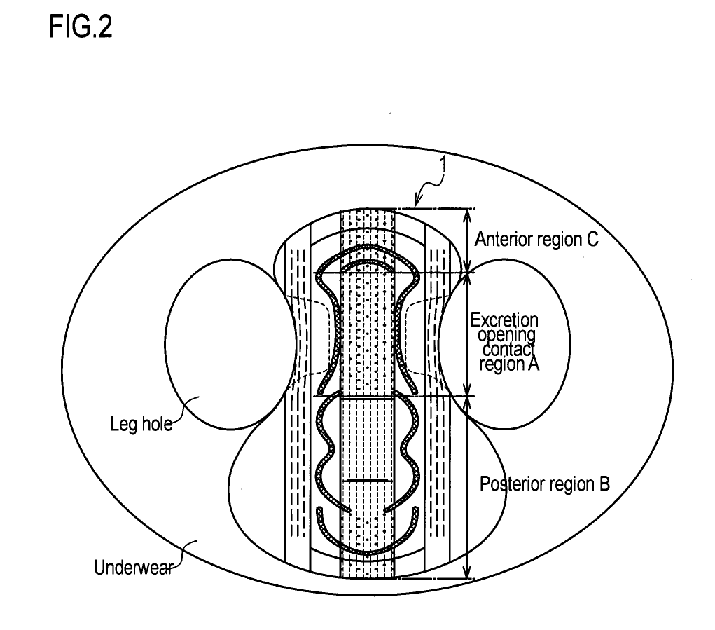

The excretion opening contact region A is a region abutting a vaginal opening of a wearer in the absorbent article 1, and is a region disposed between two leg holes of an underwear when the absorbent article 1 is attached to the underwear of the wearer, as shown in FIG. 2, for example.

In the excretion opening contact region A, the absorbent article 1 includes a pair of central wing portions 40 formed to protrude to opposite sides of a width direction W, and in the posterior region B, the absorbent article 1 includes a rear wing portion 41 formed to protrude outwardly of the width direction W.

In this case, when attaching the absorbent article 1 to the underwear, the wearer rolls the central wing portion 40 up to the underwear so that the absorbent article 1 is fixed to the underwear. Further, when attaching the absorbent article 1 to the underwear, the wearer fixes the absorbent article 1 while the rearward wing portion 41 is opened to a back neck drop of the underwear.

In the absorbent article 1, an intersection Q between a line O of the longitudinal direction L passing the center of the width direction W and a line (a-a) of a width direction W passing the center of the longitudinal direction L of the central wing portion 40 may be a “center of the longitudinal direction L of the excretion opening contact region A”, a region between a location apart by 40 mm from the intersection Q to a rearward of the longitudinal direction L and a rearward end of the longitudinal direction L may be a “posterior region B”, and a region between a location apart by 40 mm from the intersection Q to a forward of the longitudinal direction L and a forward end of the longitudinal direction L may be an “anterior region C”.

In this case, when compression grooves 14A are disposed to be curved inwardly of the width direction W along the longitudinal direction L in the absorbent article 1 in which no central wing portion 40 is provided, a line linking locations at the innermost position of the width direction W in the both compression grooves 14A may be the above-described line (a-a).

Moreover, when the compression grooves 14A are disposed to be curved outwardly of the width direction W along the longitudinal direction L in the absorbent article 1 in which no central wing portion 40 is provided, a line linking locations at the outermost positions of the width direction W in the both compression grooves 14A may be the above-described line (a-a).

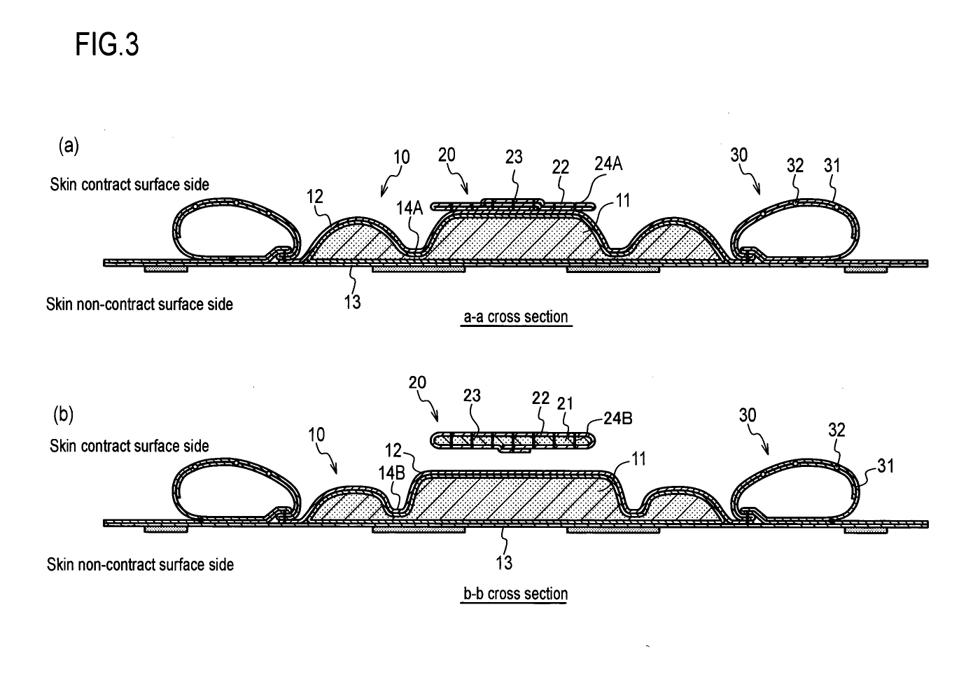

In the absorbent article 1 of this embodiment, as shown in FIG. 3(a) and FIG. 3 (b), the main body layer 10 includes a topsheet 12, a backsheet 13, and a first absorber 11 disposed between the topsheet 12 and the backsheet 13.

Conventional materials may be used for the topsheet 12, the backsheet 13, and the first absorber 11. For example, a material of the topsheet 12 is not particularly limited as long as it is a sheet material with a liquid-permeable structure, such as a nonwoven fabric, a woven fabric, a porous plastic sheet, a mesh sheet, or the like. Any natural fiber and chemical fiber may be used as a material of the woven fabric and the nonwoven fabric. The backsheet 13 may be a film mainly made of polyethylene, polypropylene or the like, an air-permeable resin film, or a sheet obtained by joining an air-permeable resin film with a nonwoven fiber such as spunbond or spunlace. The first absorber 11 may be formed by laminating a hydrophilic fiber or powder by an air-laid method, or may be an air-laid sheet which is formed in such a manner that a hydrophilic fiber or powder is formed to have a sheet shape by the air-laid method.

The topsheet 12 is a liquid-permeable sheet provided on the skin contact surface side of the absorbent article 1, in the main body layer 10, and the backsheet 13 is a liquid-impermeable sheet provided on a skin non-contact surface side of the absorbent article 1, in the main body layer 10.

The first absorber 11 is configured to absorb bodily liquids such as urine and menstrual blood, and disposed over the excretion opening contact region A, the posterior region B, and the anterior region C in the absorbent article 1.

It is noted that as shown in FIG. 3(a) and FIG. 3(b), the topsheet 12 and the first absorber 11 are joined by the compression grooves 14A and 14B extending in the longitudinal direction L.

In the absorbent article 1 of this embodiment, the upper layer 20 is separated from the main body layer 10, and is disposed farther inwardly of the skin contact surface side of the absorbent article 1 than the main body layer 10, as shown in FIG. 3(a) and FIG. 3(b). Moreover, as shown in FIG. 1, the upper layer 20 is disposed over the entire length of the product of the longitudinal direction L of the absorbent article 1.

As shown in FIG. 3(a) and FIG. 3(b), the upper layer 20 includes a second absorber 21 and a liquid-permeable sheet 22. The second absorber 21 may be formed of the same material as the first absorber 11, and the liquid-permeable sheet 22 may be formed of the same material as the topsheet 12.

As shown in FIG. 1, FIG. 3(a), and FIG. 3(b), in the upper layer 20, the second absorber 21 is not disposed in the excretion opening contact region A, but disposed in the posterior region B.

In this case, in the width direction W of the absorbent article 1, the first absorber 11 is configured to be longer than the second absorber 21. A coloring process or a pattern print process may be performed on the second absorber 21 in order for the wearer to easily identify the second absorber 21.

In the upper layer 20, farther on the skin contact surface side of the absorbent article 1 than the second absorber 21, the liquid-permeable sheet 22 is disposed over the excretion opening contact region A, the posterior region B, and the anterior region C.

Specifically, the liquid-permeable sheet 22 is configured to envelope the entire second absorber 21 by folding both regions including ends of the width direction W of the liquid-permeable sheet 22 toward a center line side of the width direction W and a main body layer 10 side, in the upper layer 20.

For example, in the upper layer 20, the liquid-permeable sheet 22 is folded in three, and regions of the liquid-permeable sheet 22 folded in three are joined by hot melt adhesive (HMA).

Then, as shown in FIG. 3 (b), in the upper layer 20 corresponding to the posterior region B, the second absorber 21 is disposed inside the liquid-permeable sheet 22 folded in three.

That is, as shown in FIG. 3(a), on the upper layer 20 corresponding to the excretion opening contact region A (and the anterior region C), the second absorber 21 is not disposed inside the liquid-permeable sheet 22 folded in three.

In this case, in the upper layer 20, an isolation region (slide unit) 20B may be provided between a region where the second absorber 21 is not disposed (i.e., a region between a boundary line 20A1 of the width direction W and the forward end of the absorbent article 1) and a region 20C where the second absorber 21 is disposed (i.e., a region between a boundary line 20A2 of the width direction W and a boundary line 20A3 of the width direction W).

In such a case, in the region 20C where the isolation region 20B and the second absorber 21 are disposed, the upper layer 20 and the main body layer 10 are not joined. On the other hand, in the excretion opening contact region A and the anterior region C, the upper layer 20 and the main body layer 10 are joined by a compressing portion (embossing) and hot melt adhesive, etc. In the posterior region B, the upper layer 20 and the main body layer 10 may be joined by the compressing portion (embossing) and hot melt adhesive, etc., in a region other than the region 20C where the isolation region 20B and the second absorber 21 are disposed.

It is noted that in the isolation region 20B, a plurality of compression grooves may be provided laterally all over the width direction W.

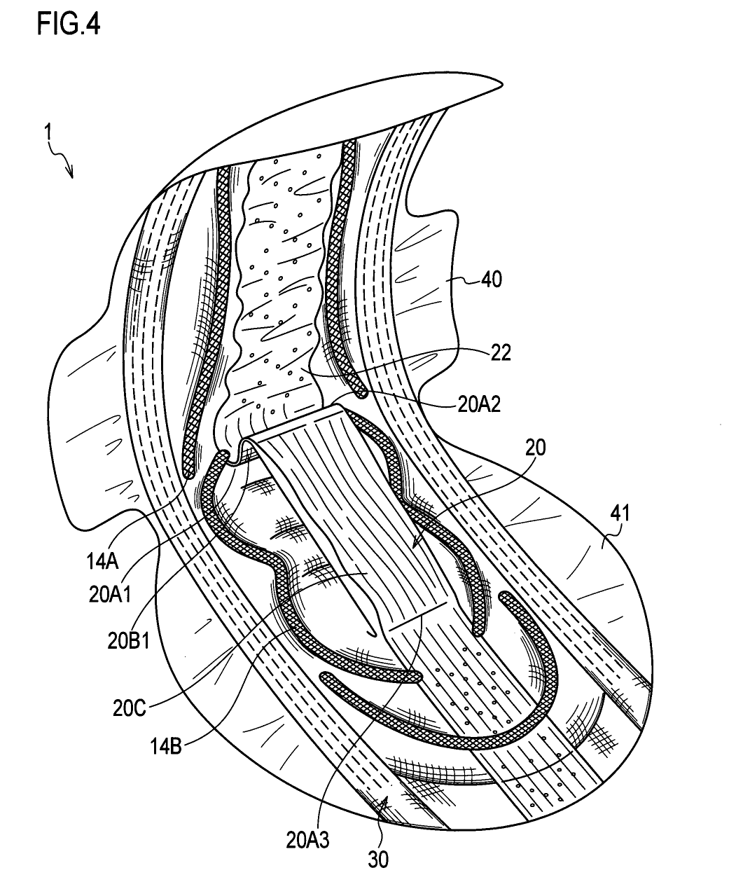

In such a case, as shown in FIG. 4, when the absorbent article 1 is attached to the underwear, the slide unit 20B is configured to stand upright toward the front of the longitudinal direction L because absorbent article 1 is curved inwardly along with the longitudinal direction L.

On the upper layer 20, the above-described isolation region 20B may not be arranged. In such a case, the upper layer 20 and the main body layer 10 may be joined over the entire length of the product of the longitudinal direction L of the absorbent article 1.

In such a case, the forward end of the longitudinal direction L of the second absorber 21 overlaps the forward end of the longitudinal direction L of the posterior region B, and the rearward end of the longitudinal direction L of the second absorber 21 is disposed further forwardly of the longitudinal direction L than the rearward end of the longitudinal direction L of the first absorber 11. According to such a configuration, the bodily liquid not absorbed by the second absorber 21 can be absorbed by the first absorber 11.

In this case, in the width direction W of the absorbent article 1, the liquid-permeable sheet 22 is configured to be shorter than the first absorber 11.

In the region where the central wing portion 40 is arranged, the both edges of the upper layer 20 are disposed not to protrude outwardly from the two compression grooves 14A in the width direction W of the absorbent article 1.

In the region where the second absorber 21 is not disposed, the compressing portion 24A is arranged on the liquid-permeable sheet 22, and by the compressing portion 24A, the regions of the folded liquid-permeable sheet 22 are joined.

In the region 20B where the second absorber 21 is disposed, a compressing portion 24B is arranged on the liquid-permeable sheet 22, and by the compressing portion 24B, the region of the folded liquid-permeable sheet 22 and the second absorber 21 are joined.

In this case, the configuration may be such that one portion of the region on the skin contact surface side of the absorbent article 1 of the second absorber 21 and the region on the skin contact surface side of the absorbent article 1 of the liquid-permeable sheet 22 are just joined by the compressing portion 24B.

According to such a configuration, it is possible to increase a movement velocity of the bodily liquid from the liquid-permeable sheet 22 to the second absorber 21 while preventing the absorbent article 1 from being hardened.

The configuration may be such that in the region 20B where the second absorber 21 is disposed, a bulk recovery process as described later is not performed. According to such a configuration, the movement velocity of the bodily liquid from the liquid-permeable sheet 22 to the second absorber 21 can be increased by lowering the bulk of the region 20B where the second absorber 21 is disposed than the bulk of the other regions.

The configuration may be such that the fabric density in the liquid-permeable sheet 22 is lower than that in the topsheet 12.

For example, it may be possible to lower the fabric density of the liquid-permeable sheet 22 compared to the topsheet 12 by whether the bulk recovery process is performed or not or changing conditions in the bulk recovery process such as a temperature, a flow rate, and a time.

The fabric density of the topsheet 12 and the liquid-permeable sheet 22 can be measured by, for example, in such a measurement method as; (1) measuring the thickness and the area of the sheet of which density is to be measured, (2) measuring the weight of the sheet of which density is to be measured, (3) calculating the base weight based on the weight of the sheet and the area of the sheet, and (4) calculating the density based on the base weight and the thickness. Moreover, the thickness of the sheet can be measured by, for example, in such a measurement method as; (1) impregnating the sheet with liquid nitrogen and freezing the sheet, (2) cutting the frozen sheet with a razor and then bringing the cut sheet to room temperature to thaw, and (3) measuring the thickness of the sheet at the cut surface with a scanning electron microscope (Keyence VE-7800).

In the absorbent article 1, leakage-preventing walls 30 may be arranged at the both ends of the width direction W of the main body layer 20. Each leakage-preventing wall 30 is configured by a leakage-preventing sheet 31 folded in midair and an elastic member 32 disposed in an expanded state in the leakage-preventing sheet 31.

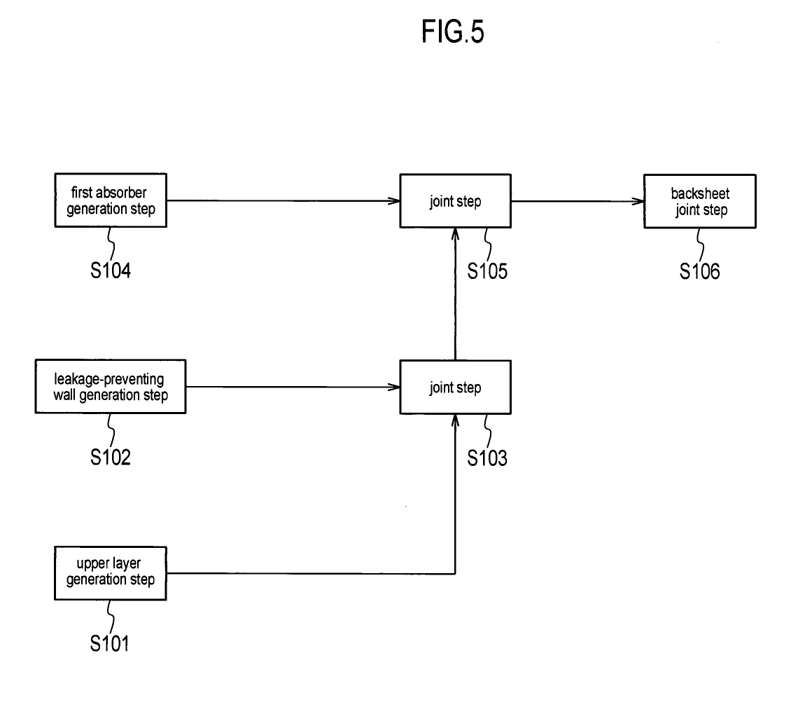

Subsequently, with reference to FIG. 5, a method of manufacturing the absorbent article 1 according to the embodiment will be partially explained. When steps not described in FIG. 5 are executed, the existing steps can be introduced.

As shown in FIG. 5, in step S101, the upper layer 20 is generated.

Specifically, firstly during one or two folding steps, the second absorber 21 is folded, and by way of temporal tacking embossing or hot melt adhesive, the temporal tacking is performed for a purpose of sealing.

Secondly, a bulk recovery process or a cooling process is performed on a web for the liquid-permeable sheet 22 so that the bulk is recovered. For example, when the web for the liquid-permeable sheet 22 is contacted with a heat roller, or hot blast is applied to the web for the liquid-permeable sheet 22 followed by the rapid cooling, the bulk of the web for the liquid-permeable sheet 22 may be recovered.

Thirdly, the folded second absorber is cut into a predetermined length, which is applied the hot melt adhesive, and the resultant product is pasted on the web for the liquid-permeable sheet 22.

Fourthly, the hot melt adhesive is applied on the web for the liquid-permeable sheet 22 and the second absorber 21, which are folded into three in the sailor step, and then the second absorber 21 is wrapped with the web for the liquid-permeable sheet 22.

Fifthly, the web for the liquid-permeable sheet 22 is embossed to form the slide unit 20C.

In step S102, the leakage-preventing wall 30 is generated.

Specifically, firstly, the hot melt adhesive is applied to the elastic member 32, which is merged to the leakage-preventing sheet 31 cut in half by a slitter so as to form the foundation of the leakage-preventing wall 30 via the sailor step.

Secondly, the foundation of the leakage-preventing wall 30 is merged to a web for the topsheet 12, and the foundation of the leakage-preventing wall 30 and the web for the topsheet 12 are joined by embossing.

Thirdly, by the sailor step, a Ω-shaped leakage-preventing wall 30 is formed from the foundation of the leakage-preventing wall 30.

In step S103, the hot melt adhesive is intermittently applied to the web for the liquid-permeable sheet 12 folded into three, which is followed by pasting on the web for the topsheet 12 on which the leakage-preventing wall 30 is formed. Then, a web for the liquid-permeable sheet 22 and the web for the topsheet 12 are joined by embossing.

In step S104, the first absorber 11 is generated.

Specifically, firstly, pulp crashed on a tissue paper and absorbent polymer are laminated, which is followed by wrapping with the tissue paper in the sailor step, thereby forming the foundation of the first absorber 11.

Secondly, the thickness of the foundation of the first absorber 11 is set to a predetermined thickness by embossing.

Thirdly, by using an absorber cutter, the foundation of the first absorber 11 is cut into a predetermined dimension and shape to generate the first absorber 11.

In step S105, the first absorber 11 is pasted on the web for the topsheet 12 to which the web for the liquid-permeable sheet 22 and the leakage-preventing wall 30 are joined, and on the skin contact surface side of the absorbent article 1, a compression groove of a predetermined shape is formed by a hinge seal.

In step S106, the hot melt adhesive is applied to a web for the backsheet 13 so as to be merged on the skin contact surface side of the absorbent article 1 of the web for the topsheet 12.

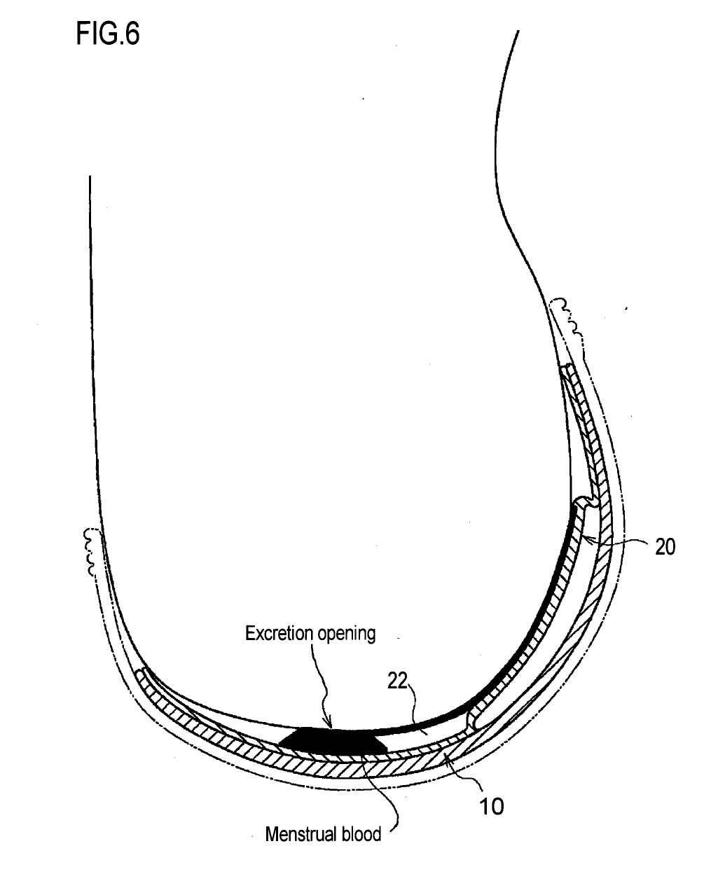

According to the embodiment-based absorbent article 1, when the wearer of the absorbent article 1 lies face up, as shown in FIG. 6, the first absorber 11 of the main body layer 10 disposed in the excretion opening contact region A can absorb the bodily liquid excreted from an excretion opening, and the second absorber 21 of the upper layer 20 disposed in the posterior region B can absorb the bodily liquid flowing along wearer' gluteal groove.

That is, there is only a slight possibility that the second absorber 21 directly touches the bodily fluid excreted from the excretion opening, and thus, the second absorber 21 is able to concentrate on the absorption of the bodily liquid flowing along the wearer' gluteal groove, etc., and is able to decrease the absorption leakage of the bodily liquid flowing along the wearer' gluteal groove, etc.

According to the embodiment-based absorbent article 1, when the regions of the folded liquid-permeable sheet 22 are increased in density by embossing in dotted pattern, etc., in the upper layer 20, for a purpose of integration, it is possible to facilitate the movement of the bodily liquid retained between the regions of the liquid-permeable sheet 22 from the upper layer 20 side to the main body layer 10 side and possible to retain the bodily liquid between the fabrics of the liquid-permeable sheet 22 while the bodily liquid moves from the upper layer 20 side to the main body layer 10 side.

According to the embodiment-based absorbent article 1, after the upper layer 20 and the main body layer 10 are joined by the hot melt adhesive, the resultant materials are partially joined by embossing in dot. In this way, the upper layer 20 and the main body layer 10 are partially increased in density for a purpose of integration. As a result, it is possible to facilitate the movement of the bodily liquid retained on the upper layer 20 side to the main body layer 10 side.

According to the embodiment-based absorbent article 1, in the posterior region B, the liquid-permeable sheet 22 and the second absorber 21 are partially joined by embossing in dot, and thus, it is possible to facilitate the movement of the bodily liquid from the liquid-permeable sheet 22 to the second absorber 21 and possible to stop the bodily liquid flowing along the irregularities formed by the embossing.

According to the embodiment-based absorbable article 1, the fabric density in the liquid-permeable sheet 22 is configured to be lower than that in the topsheet 12.

Therefore, the liquid-permeable sheet 22 low in fabric density is configured to save the bodily liquid between the fabrics until the first absorber 11 in the main body layer 10 absorbs the bodily liquid, and the resultant bodily fluid is drawn to the topsheet 12 high in fabric density, and thus, even when a large amount of bodily liquids are excreted once, it is possible to decrease the absorption leakage of the bodily liquids.

According to the embodiment-based absorbable article 1, in the upper layer 20, the fabric density of the liquid-permeable sheet 22 in the region where the second absorber 21 is disposed is configured to be higher than that of the liquid-permeable sheet 22 in the region where the second absorber 21 is not disposed, that is, the second absorber 21 on the skin contact surface side of the absorbent article 1 is covered with the liquid-permeable sheet 22 low in bulk, and thus, the performance of drawing the bodily fluid is increased.

According to the embodiment-based absorbable article 1, in the upper layer 20, the fabric density of the liquid-permeable sheet 22 in the region where the second absorber 21 is disposed is configured to be higher than that of the liquid-permeable sheet 22 in the region where the second absorber 21 is not disposed, that is, the second absorber 21 on the skin contact surface side of the absorbent article 1 is covered with the liquid-permeable sheet 22 low in bulk.

Thus, the present invention has been explained in detail by using the above-described embodiments; however, it is obvious that for persons skilled in the art, the present invention is not limited to the embodiments explained herein. The present invention can be implemented as a corrected, modified mode without departing from the gist and the scope of the present invention defined by the claims. Therefore, the description of the specification is intended for explaining the example only and does not impose any limited meaning to the present invention.

The present invention described above may be arranged in at least the following items: (i) isolation region 20B may stand upright toward the front of the longitudinal direction, (ii) the upper layer 20 and the main body layer 10 are joined over the entire length of the product of the longitudinal direction L of the absorbent article Grooves 14A, 14 B, (iii) center of wings 40 is the center of region A in direction L.

Note that this application claims the benefit of Japanese Application No. 2010-067086 the entire disclosure of which is incorporated by reference herein.

1. An absorbent article, comprising:
a main body layer including a topsheet, a backsheet, and a first absorber disposed between the topsheet and the backsheet; and
a second absorber separated from the main body layer, wherein:
the first absorber is disposed over an excretion opening contact region in the absorbent article and a posterior region of the excretion opening contact region in a longitudinal direction of the absorbent article;
in a width direction of the absorbent article, the first absorber is configured to be longer than the second absorber;
the second absorber is disposed farther inwardly of a skin contact surface side of the absorbent article than the main body layer;
the second absorber is not disposed in the excretion opening contact region but disposed in the posterior region;
a liquid-permeable sheet is disposed farther inwardly of the skin contact surface side of the absorbent article than the second absorber over the excretion opening contact region and the posterior region; and
in the width direction, the liquid-permeable sheet is configured to be shorter than the first absorber.

2. The absorbent article according to claim 1, wherein a compressing portion is arranged in the liquid-permeable sheet.

3. The absorbent article according to claim 1, wherein a fabric density in the liquid-permeable sheet is configured to be lower than a fabric density in the topsheet.

4. The absorbent article according to claim 1, wherein in the posterior region, the main body layer and the second absorber are not joined.

5. The absorbent article according to claim 1, wherein the liquid-permeable sheet envelopes the entire second absorber by folding both regions including ends of the width direction of the liquid-permeable sheet toward a center line side of the width direction and the main body layer side.
